(19) United States
(12) Patent Application Publication
(10) Pub. No.: US 2003/0195357 A1
(43) Pub. Date: Oct. 16, 2003

(54) Ruthenium complexes containing carboids

(76) Inventors: Wolfram Stuer, Mannheim (DE); Jorn Karl, Mannheim (DE); Michael Roper, Wachenheim (DE); Stefan Jung, Hochberg (DE); Justin Wolf, Weikersheim (DE); Helmut Werner, Wurzburg (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/344,367

(22) PCT Filed: Aug. 10, 2001
(86) PCT No.: PCT/EP01/09295

(30) Foreign Application Priority Data
Aug. 11, 2000 (DE) 10039389.6

(51) Int. Cl. C07F 17/02; C07F 15/00; C07C 2/02

(52) U.S. Cl. 546/2; 556/136; 585/520

(57) ABSTRACT

In the ruthenium complexes of the general formula A or B,

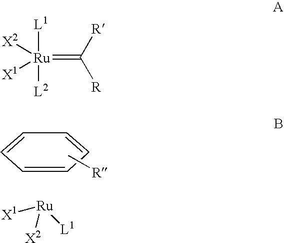

X¹ and X², independently of one another, are monodentate or polydentate anionic ligands,

R, R′ and R″, independently of one another, are hydrogen or substituted or unsubstituted C₁₋₂₀-alkyl, C₆₋₂₀-aryl or C₇₋₂₀-alkylaryl radicals, and

L¹ and L², independently of one another, are neutral electron donor ligands which are coordinated as carbenoids to the metal center and may be linked via a bridge W having 0 to 20 carbon atoms, which may be part of a cyclic or aromatic group and may be interrupted by heteroatoms, with the exception of C,N-heterocyclic five-membered ring systems.

The present invention relates to carbenoid-containing ruthenium complexes which can be employed, for example, as catalysts in metathesis reactions, and to a process for their preparation.

In its simplest form, olefin metathesis (disproportionation) describes the reversible, metal-catalyzed trans-alkylidenation of olefins by breaking and re-formation of C═C double bonds. In the case of the metathesis of acyclic olefins, a distinction is made, for example, between self-metathesis, in which an olefin is converted into a mixture of two olefins of different molar mass (example: propene→ethene+2-butene), and cross- or co-metathesis, which describes a reaction between two different olefins (propene+1-butene→ethene+2-pentene). If one of the reaction partners is ethylene, the term ethenolysis is generally used. Further areas of application of olefin metathesis are the synthesis of unsaturated polymers by ring-opening metathesis polymerization (ROMP) of cyclic olefins and acyclic diene metathesis polymerization (ADMET) of α,ω-dienes. More recent applications relate to the selective ring opening of cyclic olefins using acyclic olefins and ring closure reactions (RCM), by means of which unsaturated rings of various ring size can be prepared—preferably from α,ω-dienes.

In principle, suitable catalysts for metathesis reactions are homogeneous and heterogeneous transition-metal compounds, in particular those from sub-group VI-VIII of the Periodic Table of the Elements, and homogeneous and heterogeneous catalyst systems comprising these compounds.

In recent years, increasing efforts have been made to prepare homogeneous catalysts which are stable in protic media and in atmospheric oxygen. DE-A-197 36 609 describes alkylideneruthenium compounds of the general composition [RuX₂(═CHR)(PR′₃)₂] (R=R′=alkyl or aryl) and methods for the synthesis of complexes of this type.

The catalysts of the general formula [RuCl₂(═CHR)L₂] are very active in the metathesis of numerous olefins for L=PCy₃. In particular in the case of olefins containing polar functional groups, such as —OH, —CO₂R, —CN, etc., some of these catalysts may be deactivated rapidly. The activity of the catalysts and the deactivation rate are highly dependent on the olefin. The degree of substitution of the double bond and the position of functional groups relative to the double bond play a considerable role.

Recently, the phosphine ligands have been replaced by heteroatom-substituted carbenes as ligands. In free form, these have an electron sextuplet on a carbon atom.

DE-A-198 15 275 described N-heterocyclic carbenes as complex ligands whose ring is derived from imidazole or triazole. The complexes conform to the general formula [RuX¹X²L¹L²(═CR″R′)], where at least one of the ligands L¹ and L² is an N-heterocyclic carbene.

These catalysts containing N-heterocyclic carbenes as ligands are for some substrates superior to the catalysts containing phosphine ligands, a strong substrate dependence being evident. However, the catalysts do not allow their structure to be varied significantly. The object of synthesizing stable catalysts having a long service life for the metathesis of numerous different olefins can, however, not be achieved in this way. On the basis of experience hitherto, it is to be expected that to this end the catalyst will have to be matched to the respective substrate. This “tuning” of the catalyst is usually carried out by varying the substituents within a class of ligands. The disadvantage of N-heterocyclic carbenes consists in defining the organic basic skeleton which stands in the way of broad catalyst screening. Due to the C,N 5-membered ring structure, the angle included by the carbene carbon atom with its two adjacent atoms in the 5-membered ring is subject to narrow limits. For this reason, the space requirement of the ligands can be controlled virtually exclusively via the substituents on the last-mentioned adjacent atoms.

It is an object of the present invention to develop ligand basic structures which allow wide-ranging variation of the substituents and of the skeleton in order to facilitate variable catalyst design. The aim is for it to be possible to vary the steric conditions and the electronic conditions in a wide-ranging manner. The aim was to find generally valid syntheses which can be applied to a large number of starting materials and thus allow the synthesis of a large number of ligands. A further aim is for the requisite starting materials to be as far as possible commercially available or easy to prepare. In order to achieve high throughput, it should be possible to transfer the synthesis to an automatic synthesizer in order to facilitate automatic build-up of a ligand library and thus a catalyst library. This should make it possible to be able to optimize ruthenium metathesis catalysts specifically for a substrate.

We have found that this object is achieved by ruthenium complexes of the general formula A or B

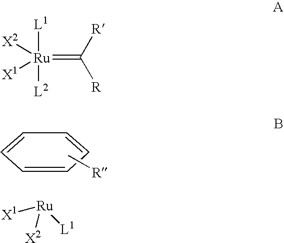

where

X¹ and X², independently of one another, are monodentate or polydentate anionic ligands,

R, R′ and R″, independently of one another, are hydrogen or substituted or unsubstituted C₁₋₂₀-alkyl, C₆₋₂₀-aryl or C₇₋₂₀-alkylaryl radicals, and

L¹ and L², independently of one another, are neutral electron donor ligands which are coordinated as carbenoids to the metal center and may be linked via a bridge W having 0 to 20 carbon atoms, which may be part of a cyclic or aromatic group and may be interrupted by heteroatoms, with the exception of C,N-heterocyclic five-membered ring systems.

The neutral electron donor ligands L¹ and L² preferably, independently of one another, have the general formula C

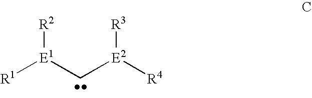

where

R¹ to R⁴, independently of one another, are electron pairs, hydrogen or substituted or unsubstituted C₁₋₂₀-alkyl, C₆₋₂₀-aryl or C₇₋₂₀-alkylaryl radicals, where (R¹ and R²) and/or (R² and R³) and/or (R³ and R⁴) together may form a cyclic radical, and

E¹ and E², independently of one another, are elements from the group consisting of B, CR⁵, SiR⁵, where R⁵ is as defined for R¹ to R⁴, N, P, As, Sb, O and S, corresponding to their valency.

The neutral electron donor ligands L¹ and L² are particularly preferably selected, independently of one another, from cyclic and acyclic diaminocarbenes (I, II where n≧1, and III), aminooxycarbenes (IV), bisoxycarbenes, aminothiocarbenes (V), aminophosphinocarbenes, phosphinooxycarbenes (VII), phosphinophosphino-carbenes (VIII), phosphinosilylcarbenes (IX) and diborylcarbenes (X), where the ligands L¹ and L² may also be linked to one another by the bridge W and can thus form a chelate ligand

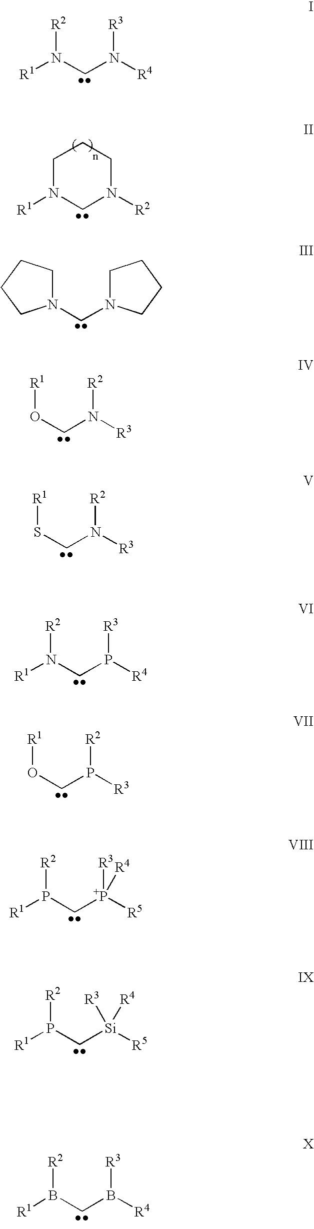

The anionic ligands are preferably weakly or non-coordinating anions, for example ClO₄⁻, PF₆⁻, BF₄⁻, BAr₄⁻ or sulfonate.

The electronic properties of the carbene carbon atom may be substantially controlled by the variable substitution by identical or different fragments ER¹R² or E²R³R⁴. Thus, the electron deficiency in dianinocarbenes is reduced by the π-donor, σ-acceptor character on the NR₂ fragments. In diborylcarbenes, by contrast, the electron deficiency of the carbon atom is increased by the boron atoms acting as π-acceptors and σ-donors. In between these are, for example, phosphonosilylcarbenes (cf. Chem. Rev. 2000, 100, 39-91). The properties of the catalyst can thus be varied via the coordination to the transition metal ruthenium.

The invention furthermore relates to the use of these catalyst systems in metathesis reactions of olefins. Compared with alkylideneruthenium(II) complexes of the type [RuCl₂(═CHR)L₂] known from the literature, which, as homogeneous metathesis catalysts, have a high application potential, the abovementioned compounds are distinguished by significantly broadened variability of the structures and by simple preparation of the property-determining ligands L¹ and L².

On use as metathesis catalysts, the complexes of type A or B can either react with the olefin without activation or can be activated in situ using acids HX* or using light, where X* is, for example, CF₃CO₂⁻or CF₃SO₃⁻.

In contrast to the known ruthenium-containing catalyst systems, the ligands employed in the catalysts according to the invention can be prepared to a wide extent with different structures with the aid of automatic synthesizers. It is thus possible to prepare large ligand and catalyst libraries in an automated manner. Ligands and catalysts according to the invention allow substantial variation from a steric and also electronic point of view. This enables the preparation of a large number of catalysts having different properties, which can then be subjected to catalyst screening and tuning for a specific application in a certain reaction. For example, an intentional reaction can be carried out in parallelized form in a multiplicity of reactors using different catalysts from the catalyst library, with it being possible to vary specifically the catalysts recognized as the most active or selective. Corresponding combinatorial or automated preparation processes using automatic synthesizers for this purpose are known, see, for example, A.M. La Pointe, J. Comb. Chem. 1999, 1, 101-104.

The ruthenium complexes according to the invention can be prepared by any desired suitable processes, as carried out, for example, in the specifications cited above.

The invention thus relates to a process for the preparation of the ruthenium complexes according to the invention by reaction of ruthenium complexes of the general formula [RuHX¹(H₂)L*L**] with the free ligands L¹ and L² and acids HX₂, or salts thereof, and alkynes or R″—C₆H₅, where L* and L** are neutral two-electron donors.

In addition, the invention relates to a process for the preparation of the ruthenium complexes by reaction of RuCl₃.xH₂O or [RuCl₂(olefin)]₂ or [RuCl₂(COD)]ₙ with the free ligands L¹ and L² or with the salts [HL^{]X}¹ and [HL²]X² in the presence of a base and hydrogen to give precursor compounds, which are themselves reacted with alkynes and acids HX¹ and HX².

In addition, the invention relates to a process for the preparation of ruthenium complexes B by the reaction of [RuCl₂(arene)]₂ or [(arene)RuCl₂(L*)]₂ with the free ligand L¹ or the salt [HL¹]X¹ in the presence of a base, where L* is a neutral two-electron donor.

This process can be carried out in an automated manner in parallel in a plurality of reaction vessels for the preparation of a plurality of different ruthenium complexes A and/or B.

The active components A and/or B can be synthesized starting from numerous organometallic starting materials, for example

by reaction of carbene complexes of the composition [RuX¹X²(═CRR′)L*L**] with the free carbenes of type C.

One possible starting compound for the preparation of the active component A is, for example, the compound [RuCl₂(═CHCH₃)(PCy₃)₂]. It can be prepared according to literature details by reaction of the unisolated intermediate [RuHCl(H₂)(PCy₃)₂] with 1-alkynes in the presence of HCl sources (DE-A-197 36 609). [RuHCl(H₂)(PCy₃)₂] itself is accessible, for example, from the polymeric ruthenium precursor [RuCl₂(COD)]ₓ (COD=cyclooctadiene) in i-propanol in the presence of PCy₃ under a hydrogen atmosphere (Werner et al., Organometallics 1996, 15, 1960-1962) or starting from the same starting material in sec-butanol in the presence of PCy₃ and tertiary amines (NEt₃) under a hydrogen atmosphere (Grubbs et al., Organometallics 1997, 16, 3867-3869). [RuHCl(H₂)PCy₃)₂] is furthermore accessible starting from RuCl₃.H₂O in THF by reaction with PCy₃ in the presence of activated magnesium under a hydrogen atmosphere and is preferably reacted in situ with 1-alkynes to give the corresponding hydrido-(chloro)vinylidene complexes [RuClH(═C═CHR)(PCy₃)₂]. The latter can be isolated or react in situ with HCl sources to give [RuCl₂(═CHCH₃)(PCy₃)₂]. The last-mentioned compound is reacted with the free carbene ligands of type C to give the active component A according to the invention, with one equivalent of PCy₃ being cleaved off. The preparation of the free ligands of type C is described in the review article by Bourissou et al. (Chem. Rev. 2000, 100, 39-91) and the literature cited therein.

Carbenes of type C can be prepared, for example, by the following reaction sequence. The diaminocarbenes of type I and III can be synthesized as follows. Sequences of this type can be carried out by automatic synthesizers. Owing to the large number of commercial starting materials, this allows the synthesis of a wide variety of carbene ligands of type C.

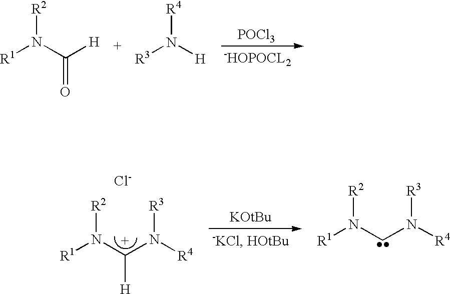

For symmetrical diaminocarbenes, the following sequence can also be employed:

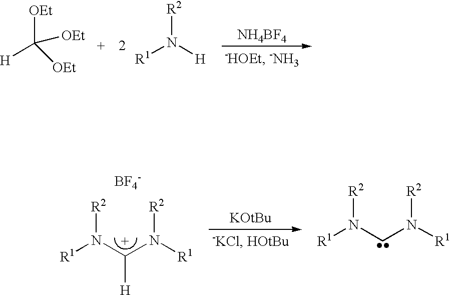

The reaction of free carbenes with carbene complexes of the type [RuX¹X²(═CRR′)L*L**] has been described for N-heterocyclic carbenes: Hermann et al. in Angew. Chem. 1998, 110, 2631-2633, Angew. Chem. 1999, 111, 2573-2576, DE-A-198 15 275, Grubbs et al. Tetrahedron Lett. 1999, 40, 2247-2250, Organic Lett. 1999, 1, 953-956; Nolan et al. J. Am. Chem. Soc. 1999, 121, 2674-2678, and can be carried out in a similar manner for the carbenes of type C. The reactions are advantageously carried out in an automatic synthesizer.

By reaction of arene complexes of the type [areneRuX¹X²L¹] with free carbenes of type C.

Areneruthenium complexes, such as [(p-cymol)RuCl₂(PPh₃)] are obtained by stirring the dimeric starting materials with PPh₃. Thus, [(p-cymol)RuCl₂]₂ reacts with PPh₃ in organic solvents to give [(p-cymol)RuCl₂(PPh₃)]. The last-mentioned compound or a dimer such as [(p-cymol)RuCl₂]₂ is reacted with the free carbene ligands of type C to give the active components B according to the invention, with one equivalent of PPh₃ being cleaved off.

By reaction of the compounds of the type [RuX¹X²(═CRR′)L¹L²] or [areneRuX¹X²L¹] with the carbenes of type C generated in situ.

The carbenes of type C can be generated in the presence of the organometallic starting material by reaction of the carbene precursors [L¹H³⁰ ]Y or [L²H⁺]Y⁻ with strong bases, for example KOtBu or LDA (lithium diisopropylamide) and react directly to give the active components A and/or B without being isolated in advance.

Reactions to give the active components A and/or B are carried out in organic solvents under an inert-gas atmosphere. The reaction is preferably carried out in THF or toluene or mixtures of the two at temperatures of from −100 to +100° C., preferably from 0 to 100° C., and pressures of from 1 mbar to 100 bar, preferably from 0.5 to 5 bar.

The reaction can be carried out with one or more mole equivalents of C or precursors of C. The resultant compositions comprising the active components A and/or B can be employed in situ as highly active metathesis catalyst system or isolated and stored under an inert-gas atmosphere. If desired, the active components A and B are employed in isolated form.

In general, the reaction of the substances of the general structure C or precursors thereof with suitable ruthenium complexes to give A or B is complete after from 1 second to 10 hours, preferably after from 3 seconds to 1 hour. Suitable reaction vessels are generally glass or steel containers, which may be lined with ceramic.

The invention furthermore relates to the use of these catalyst systems in metathesis reactions of olefins. Compared with alkylideneruthenium(II) complexes of the type [RuCl₂(═CHR)L₂] known from the literature, which, as homogeneous metathesis catalysts, have high application potential, the abovementioned compounds are distinguished by significantly broadened variability of the structures and by simple preparation of the property-determining ligands L¹ and L². In contrast to previously described systems, the catalysts can therefore easily be optimized for a certain substrate.

The catalyst complexes A and B obtained in this way can be employed, inter alia, for

self-metathesis of an olefin or cross-metathesis of two or more olefins

ring-opening metathesis polymerization (ROMP) of cyclic olefins

selective ring opening of cyclic olefins using acyclic olefins

acyclic diene metathesis polymerization (ADMET)

ring closure metathesis (RCM)

and further novel metathesis variants.

We claim:

1. A ruthenium complex of the general formula A or B 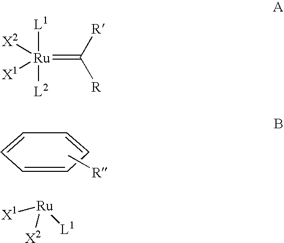
where
X¹ and X², independently of one another, are monodentate or polydentate anionic ligands,
R, R′ and R″, independently of one another, are hydrogen or substituted or unsubstituted C₁₋₂₀-alkyl, C₆₋₂₀-aryl or C₇₋₂₀-alkylaryl radicals, and
L¹ and L², independently of one another, are neutral electron donor ligands which are coordinated as carbenoids to the metal center and may be linked via a bridge W having 0 to 20 carbon atoms, which may be part of a cyclic or aromatic group and may be interrupted by heteroatoms, with the exception of C,N-heterocyclic five-membered ring systems.

2. A ruthenium complex as claimed in claim 1, wherein the neutral electron donor ligands L¹ and L² preferably, independently of one another, have the general formula C 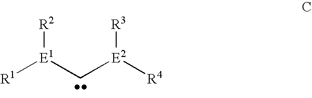
where
R¹ to R⁴, independently of one another, are electron pairs, hydrogen or substituted or unsubstituted C₁₋₂₀-alkyl, C₆₋₂₀-aryl or C₇₋₂₀-alkylaryl radicals, where (R¹ and R²) and/or (R² and R³) and/or (R³ and R⁴) together may form a cyclic radical, and
E¹ and E², independently of one another, are elements from the group consisting of B, CR⁵, SiR⁵, where R⁵ is as defined for R¹ to R⁴, N, P, As, Sb, O and S, corresponding to their valency.

3. A ruthenium complex as claimed in claim 2, wherein the neutral electron donor ligands L¹ and L² are selected, independently of one another, from cyclic and acyclic diaminocarbenes (I, II where n≧1, and III), aminooxycarbenes (IV), bisoxycarbenes, aminothiocarbenes (V), aminophosphinocarbenes, phosphinooxycarbenes (VII), phosphino-phosphinocarbenes (VIII), phosphinosilylcarbenes (IX) and diborylcarbenes (X), where the ligands L¹ and L² may also be linked to one another by the bridge W and can thus form a chelate ligand 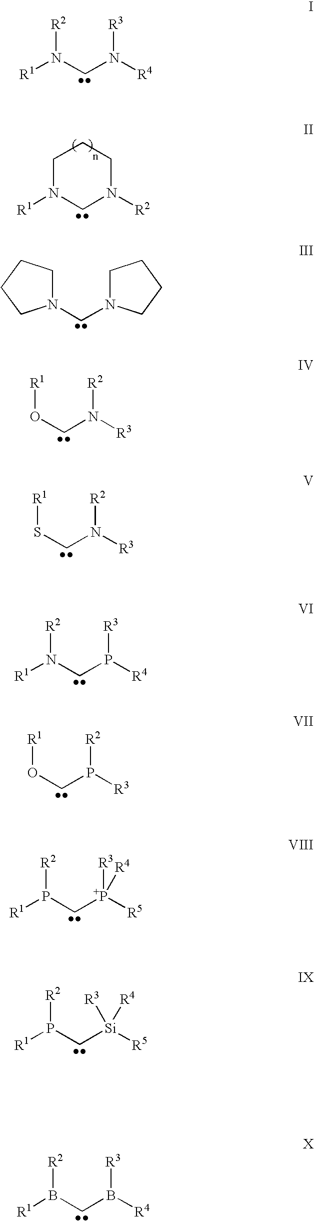
where
R¹ to R⁵, independently of one another, are electron pairs, hydrogen or substituted or unsubstituted C₁₋₂₀-alkyl, C₆₋₂₀-aryl or C₇₋₂₀-alkylaryl radicals, where (R¹ and R²) and/or (R² and R³) and/or (R³ and R⁴) together may form a cyclic radical.

4. A ruthenium complex as claimed in one of claims 1 to 3, wherein the anionic ligands are weakly or non-coordinating anions.

5. A process for the preparation of a ruthenium complex as claimed in one of claims 1 to 4 by reaction of ruthenium complexes of the general formula [RuHX¹(H₂)L*L**] with the free ligands L¹ and L² and acids HX₂, or salts thereof, and alkynes or R″—C₆H₅, where L* and L** are neutral two-electron donors,
and
X¹ and X², independently of one another, are monodentate or polydentate anionic ligands,
R″ is hydrogen or a substituted or unsubstituted C₁₋₂₀-alkyl, C₆₋₂-aryl or C₇₋₂₀-alkylaryl radical, and
L¹ and L², independently of one another, are neutral electron donor ligands which are coordinated as carbenoids to the metal center and may be linked via a bridge W having 0 to 20 carbon atoms, which may be part of a cyclic or aromatic group and may be interrupted by heteroatoms, with the exception of C,N-heterocyclic five-membered ring systems.

6. A process for the preparation of a ruthenium complex A as claimed in one of claims 1 to 4 by reaction of RuCl₃.xH₂O or [RuCl₂(olefin)]₂ or [RuCl₂(COD)]ₙ with the free ligands L¹ and L² or with the salts [HL¹]X¹ and [HL²]X² in the presence of a base and hydrogen to give precursor compounds, which are themselves reacted with alkynes and acids HX¹ and H²,
where
X¹ and X², independently of one another, are monodentate or polydentate anionic ligands,
L¹ and L², independently of one another, are neutral electron donor ligands which are coordinated as carbenoids to the metal center and may be linked via a bridge W having 0 to 20 carbon atoms, which may be part of a cyclic or aromatic group and may be interrupted by heteroatoms, with the exception of C,N-heterocyclic five-membered ring systems.

7. A process for the preparation of a ruthenium complex B as claimed in one of claims 1 to 4 by the reaction of [RuCl₂(arene)]₂ or [(arene)RuCl₂(L*)]₂ with the free ligand L¹ or the salt [HL¹]X¹ in the presence of a base, where L* is a neutral two-electron donor and X¹ is a monodentate or polydentate anionic ligand, L¹ is a neutral electron donor ligand which is coordinated as carbenoid to the metal center, with the exception of C,N-heterocyclic five-membered ring systems.

8. A process as claimed in one of claims 5 to 7, which, for the preparation of a plurality of different ruthenium complexes A and/or B, is carried out in an automated manner in parallel in a plurality of reaction vessels.

9. The use of a complex of type A or B as claimed in one of claims 1 to 4 as a catalyst for olefin metathesis reactions.

10. The use as claimed in claim 9, wherein the complexes of type A or B either react with the olefin without activation or are activated in situ by means of acids HX*, in which X* is CF₃CO₂ or CF₃SO₃, or by means of light.
